(19) United States
(12) Patent Application Publication
(10) Pub. No.: US 2021/0056419 A1
(43) Pub. Date: Feb. 25, 2021

(54) Method for Training a Deep Neural Network for Recognition of Emergency Non-Blood Treatment Alternatives to Blood Transfusion in the Jehovah's Witness Patient

(71) Applicant: Firpo Wycoff Carr, Lakewood, CA (US)

(72) Inventor: Firpo Carr, Lakewood, CA (US)

(73) Assignee: Firpo Wycoff Carr, Lakewood, CA (US)

(21) Appl. No.: 17/069,732

(22) Filed: Oct. 13, 2020

(51) Int. Cl. G06N 3/08; G06K 9/62; A61K 35/14

(52) U.S. Cl. CPC G06N 3/08; A61K 35/14; G06K 9/6256

(57) ABSTRACT

Although artificial intelligence (AI) solutions such as Natural Language Processing (NLP) or a Recurrent Neural Network (RNN) are increasingly being applied in the healthcare and medical fields, their potential to aid in critical life-threatening emergency surgeries have not been fully realized. This is attributable to underdevelopment. A method to train a deep neural network (DNN), another form of AI, has been devised to recognize emergency non-blood treatment alternatives to blood transfusion in the Jehovah's Witness patient. The official position of the Christian Congregation of Jehovah's Witnesses is that blood transfusion should be avoided at all costs, even in emergency life-or-death situations. Through sophisticated algorithms, a DNN can extend the reach of current deficient AI applications by being trained to identify blood transfusion alternative treatments for virtually every conceivable type of emergency surgery.

### BACKGROUND

Computer technology facilitates treatment in medical emergencies involving blood transfusion. In the broader scope of healthcare or bioinformatics, forms of artificial intelligence (AI), such as deep neural networks (DNN), are used to dispense healthcare, process medical procedures and treatment, and manage structured data in the form of electronic health records (EHR) and electronic medical records (EMR). More specifically, in standard of care emergency medical treatment encompassing a DNN, it is highly likely that blood transfusion would be administered since, according to the American Medical Association (AMA) and the U.S. Food and Drug Administration (FDA), blood transfusion is both the number one medical treatment in the U.S. as well as the most overutilized.

Critically, even in a life-or-death emergency, the Jehovah's Witness patient would refuse a blood transfusion, and current AI solutions are ill-equipped to offer non-transfusion alternatives or effective blood management procedures in an attempt to save the patient's life. Presently, a DNN, which has more layers of sophisticated programmed self-learning algorithms than a neural network, has not been trained to recognize alternatives to blood transfusion, hence, is algorithmically underdeveloped when it comes to processing and outputting emergency non-blood alternative surgical treatments and blood management techniques for the Jehovah's Witness patient. Also, DNNs fall short of addressing the uniqueness of Witness (or non-Witness) patient injuries in emergencies where considerable blood loss triggers the perceived need for a blood transfusion.

Applicant's invention addresses these algorithmic deficiencies by utilizing a blend of two features. The first feature trains the deep neural network through feature enhancements in the form of mathematical functions that include improving the interactive array of alternative non-blood surgical treatment options and blood management procedures through an expanded training set. For example, with a training set, the first feature enhancement enables the processing of bloodless transfusion alternatives and blood management procedures in emergency surgeries on the Jehovah's Witness patient involving but not limited to, for instance, penetrating trauma.

The deep neural network's parallel process capability is trained with this expanded training set using stochastic learning with backpropagation, which is a type of machine learning algorithm that uses the gradient of a mathematical loss function (the less loss, the better) to adjust the weights of the network. However, even with this parallel processing's algorithmic power, there is the question as to the uniqueness of each patient's penetrating trauma, for example, and the distinctiveness of the body's reaction to said trauma.

These challenges are met with the second feature of applicant's invention, which identifies comprehensive spectrum injuries to the highly vascular viscera, for example, and narrows these with taxonomic specificity by performing an iterative training algorithm, in which the system is retrained with an updated training set containing controlled nuances that are more focused. The combination of features provides more robust recognition of emergency non-blood treatment alternatives to blood transfusion in the Jehovah's Witness patient.

1. A computer-implemented method of training a deep neural network for recognition of emergency non-blood treatment alternatives to blood transfusion in the Jehovah's Witness patient: collecting blood transfusion events during surgery from a structured database; applying one or more algorithmic permutations to determine the optimum route for a blood transfusion alternative to each transfusion event; creating a first training set comprising the collected set of blood transfusion events, bloodless treatment alternatives to each event, and treatment sans blood transfusion and accompanying alternative; training the neural network in a first stage using the first training set; creating a second training set for a second stage of training comprising the first training set and blood transfusion events deemed unnecessary but perceived as being necessary, and subsequent bloodless alternatives; and training the neural network in a second stage using the second training set.
